(19) United States
(12) Patent Application Publication
(10) Pub. No.: US 2004/0220856 A1
(43) Pub. Date: Nov. 4, 2004

(54) Method of doing business that encourages the release of fish caught by anglers

(76) Inventor: Jeffrey Robert Moore, Middletown, OH (US)

Correspondence Address: Jeffrey R. Moore, 5009 Oakview Drive, Middletown, OH, 45042, US

(21) Appl. No.: 10/794,199

(22) Filed: Mar. 5, 2004

Related U.S. Application Data
(60) Provisional application No. 60/463,510, filed on Apr. 16, 2003.

(51) Int. Cl. G06F 17/60; C12N 9/00

(52) U.S. Cl. 705/14

(57) ABSTRACT

The present invention provides a means for obtaining and maintaining reasonably reliable data on individual fish in an accessible database. The present invention also includes a method of doing business that encourages returning fish caught by anglers back into the ecosystem. The method of doing business generally relates to using unique fish identification to provide data to people and organizations trying to lean more about fish. The present invention also provides a specially coded tag with a identification sequence for use with the business method. The present invention may also comprise a kit including a suitable coded tag, a means of recording the data of fish tagged and a means for reading and recording the data from previously tagged fish.

This application claims the benefit of priority under 35 U.S.C. 119(e) to provisional application 60/463,510 filed Apr. 16, 2003.

### BACKGROUND OF THE INVENTION

Sport fishing is enjoyed by millions of people every year and many fish are caught and release each year by anglers. However a significant portion of sport fishermen catch and keep the fish they have caught.

Removing the fish from the ecosystem denies future anglers the opportunity to catch and release the same fish. Larger fish are particularly susceptible to being kept. These larger fish represent an opportunity for increasing the fish population through their potential offspring, offspring that could be increased through the practice of catch and release.

Fish tagging, especially by fish hatcheries, biologists, and scientists is known. However, past tagging practice has had several limitations. Many fish die when they are young fingerlings and the tagging effort is wasted. Those tagged fish that are recovered are often removed from the ecosystem by the angler. Even if the fish is returned, the tag is often removed as evidence that the fish was caught, or to provide the data needed to specifically identify the fish caught. Even when the tagged fish is returned the data from the fish is narrowly distributed. Often, the tagging information is not provided back to the person or organization that did the initial tagging. Another limitation of previous tagging efforts is that they have not sufficiently encouraged the releasing of fish caught during recreational fishing. A method that encourages recreational and/or professional anglers to tag fish, record data on the fish and make that data available to others remains lacking in the art. The previous methods of tagging provided inadequate motivation to anglers to release the fish with the tag intact and/or record information about the fish to share with others.

Individuals and organizations have studied fish using various tags. However, the expense of tagging, the limited return rate of information and the lack of funding have all limited the use of fish tagging in spite of its value as a fish management tool.

While rewards for the return of tags in general have been contemplated, such efforts without a broader business strategy are too expensive and inefficient. A tagged fish may not be recaptured for years. Further, the tag and/or the fish are often kept to validate the data provided. A public database of tagging information has previously been incompatible with a reward system since the database would include the tag number.

A number of fish tagging mechanisms are known. All of the art disclosed herein is incorporated by reference. U.S. Pat. No. 1,742,649, issued to Eastman, discloses a band-type identification tag and method of banding fish for identification and conservation.

U.S. Pat. No. 4,790,090, issued to Sharber, discloses a fish tag having an invasive cellularly adhering point of attachment. Sharber teaches invasive anchoring of a fish tag by initial mechanical attachment and subsequent assimilation with the flesh of the fish through cellular adhesion, impregnation or ingrowth (absorption by the fish body).

U.S. Pat. No. 4,920,670, issued to Amick, discloses a fish tag including a monofilament having a retention member on one end and a flexible indicia bearing tube positioned about the monofilament.

U.S. Pat. No. 3,545,405, issued to Jefferts, discloses an identifying tag which is capable of being implanted in a macro-organism, such as a fish. The tag disclosed by Jefferts comprises a small metallic body having on their surface thereof coded information in the form of a predetermined pattern which is imparted on the surface of the body by a high-energy heat source, such as a laser.

U.S. Pat. No. 4,713,315, issued to Smith, discloses a wire tag etching system.

U.S. Pat. No. 4,750,490, issued to Haw et al., discloses a method for tagging fish for identification including an identification tag that is shallowly implanted within transparent or semi-transparent tissues of respective specimens wherein markings on the tag remain visible. Haw et al. teaches a method which includes implanting a tag, releasing the tagged fish into a body of water, subsequently capturing the fish and optically reading the detectable markings through the transparent tissue. In an alternate embodiment, Haw et al. contemplates removing the tag from the fish and reading the detectable markings upon removal of the tag.

U.S. Pat. No. 4,333,072 issued to Beigel, discloses an identification device including one method of radio frequency identification (RFID). Beigel discloses technical details that may be helpful in the manufacture of an RFID device.

U.S. Pat. No. 5,214,409 issued to Beigel, discloses an identification device including multi-memory electronic identification tags that have read only and read-write memory used and created by various means. Beigel discloses technical details that may be helpful in the manufacture of an RFID device.

U.S. Pat. No. 5,235,326 issued to Beigel et al, discloses an identification device including other sensors as part of the tag. Beigel discloses technical details that may be helpful in the manufacture of an RFID device.

Current catch and release programs and tag utilization methods lack the proper incentives to motivate people to tag fish, record the data of tagged fish, share the data with others and importantly, release the fish back into the ecosystem. When a fish is caught, the angler releasing the fish loses all contact with the fish and does not have an efficient, long-term organized means to learn about the fish's past or what may happen to “his (or her)” fish in the future. The angler also receives very little recognition for his action, especially when returning an average size fish that may one day grow to trophy proportions. Accordingly, there exists a need for a method for tracking fish, collecting data, and encouraging catch and release fishing.

There are numerous objectives of the present invention that may be achieved. Achieving even one objective may be sufficient for the business method to succeed. One objective of the present invention is to encourage the public to gain more information concerning the importance of catch and release of fish. An objective of the present invention is to encourage the release of fish caught by sport fishermen, especially the release of trophy size fish.

Another objective of the present invention is to better inform people about fish and the fishermen that release them. An objective of the present invention is to provide a method of doing business that encourages the practice of catch and release. Another objective of the present invention is to provide a method for the angler to maintain an interest in the fish by tagging it. The angler may also learn of future anglers that catch the same fish. Each angler may reinforce the benefits of catch and release to one another and help build an angling community commitment to catch and release fishing. It is a further objective to create camaraderie among catch and release anglers that can be used to encourage others to practice catch and release fishing. An objective of the present invention is to gather data on fish. It is a further objective to provide a means for encouraging communication between anglers on fishing, fishing methods and fish as a natural resource. It is also an objective of the present invention to raise the awareness of catch and release fishing in the angling community and change the way people think about fishing. At least some of the fish caught fishing, especially trophy and near trophy fish, are better tagged, and released, rather than mounted on a wall, or fried for dinner.

### SUMMARY OF THE INVENTION

The present business method includes providing an incentive to people, especially recreational and professional anglers, to release the fish they catch, and encourage other to do so as well. This incentive may be provided by a combination of fish tagging, data availability, data updating and angler communication. The method further provides generally reliable data for fish research. The method may also provide a motive for the purchase of equipment and supplies to tag fish. A motive to visit a particular web site that can provide a platform for marketing other products and services may also be provided by the method.

The present invention may include a business method for encouraging the release of fish caught by recreational anglers. The method may be used to identify the released fish. The method may also be used by people other than recreational anglers. The method may be used for the identification of birds, reptiles, and mammals where subsequent capture is possible, and the next capturer is not expected to be by the same individual or organization that completed the original capture.

The present invention may include a method of recording fish and/or fishing information such as location, weight, time, lure/bait used, weather, fishing method, water temperature depth and any other pertinent details each time the fish is caught. Information on the angler may also be part of the database. The information may include e-mail, number of fish caught that day, number of tagged fish caught, number of fish caught and/or tagged over an extended period of time, and any other desired information. Generally, this is more information than would otherwise be reliably memorized or conveniently recorded upon an appropriate size tag.

With the present invention, freshwater, saltwater, and fly fishing anglers (hereinafter angler(s)) or other individuals such as those that raise and/or release fish for stocking purposes may be provided with an apparatus and/or method to enable the angler to quickly and easily tag a fish with a tag which is attached to the fish. Preferably, the tag remains affixed for the life of the fish. Once tagged, if the fish is caught again, the information about the fish and/or the second angler may be added to the data already recorded and previously made available. The information obtained provides data about the movements and habits of the tagged fish and/or the angling community.

The apparatus according to the present invention may include a suitable fish tag having an identification sequence that can be obtained by the subsequent individual (angler) that catches the fish. The identification sequence is used to identify the fish and correlate that fish with additional data available in a searchable and updateable database. At least a portion of the identification sequence is also used as a semi-secret code to limit data entry on a fish to those that have caught the fish, and have the entire identification sequence. The data set of data on the fish is preferably part of a widely accessible database, such as an Internet accessible database that contains information on many fish tagged with a unique identification sequence. The method also has applicability to local databases such as those used by a fishing guide service, state fisheries agencies or private lakes.

In a preferred embodiment, the tag according to the present invention may be inserted into the fish. The tag is preferable non-toxic, since the fish may be eaten by other fish or predators. The identification sequence is preferably obtainable from an internal tag through the use of a device such as a reader, which does not require removing the tag.

Alternatively, the tag with the identification sequence may be external to the fish. The identification sequence may be imprinted, engraved or otherwise obtainable from the tag. The tag would include the identification sequence. The tag may also identify the internet wed address where further data can be obtained. The tag may be removed but preferably, it would remain attached to the fish. The tagging system may alternatively include a combination of an internal and external tag. The external tag may let the casual angler know that there is an internal tag. This may encourage the release of the fish and provide notice that if they chose to eat the fish, they should look for the internal tag before consumption.

The invention may include a kit. The kit may include a tag insertion device, tag reader for reading the identification sequence on the tag, a selection of tags, a data recording means, and/or combinations thereof.

### BRIEF DESCRIPTION OF THE DRAWINGS

The instant invention is described with particular reference to the following drawings.

FIG. 1A is an illustration of a fish tag in accordance with the present invention.

FIG. 1B is an illustration of a fish tag in accordance with the present invention.

FIG. 2 is an illustration of a tag being read by a tag reader.

FIG. 3 is an illustration of a fish tag in accordance with the present invention.

FIG. 4 shows a tag being inserted into a fish with an insertion device.

FIG. 5 is a sample database data screen of a particular fish.

### DETAILED DESCRIPTION OF THE INVENTION

The subheadings contained within the detailed description of the invention are intended ONLY to assist the reader. Each aspect may be closely related with other aspects of the invention, therefore a particular aspect of the present invention may be further described and disclosed elsewhere in the application. Each aspect of the present invention can be fully understood only within the entire text of the disclosure.

Definitions

As used herein the following terms are used consistent with their common usage and the following specific applications.

“Read” refers to obtaining the identification sequence.

“Scanned” refers to examining a tag by something other than the human eye, such as with a tag reader to read a tag.

“Identification sequence” is used herein to represent any combination of signals, characters, symbols, marks or other indicia that may be read by the human eye or scanned with the assistance of a machine. Indicia individually or in combination define an identification sequence, identifier, or code.

“Data” is used herein as at least one element of information. This element of information is generally part of a data set.

“Data set” is used herein as a collection of data, usually regarding a particular item of interest such as a fish. The collection of data may include many pieces of data.

“Database” is used herein as a collection of data and or data sets. The database may include many searchable and sortable data and/or data sets.

“User” refers to angler or person that catches the fish and/or the person entering data into the database.

The Tag

The tag used on the fish should remain useable for at least a year, preferably more than five years. The tag may be attached internally to the fish, external to the fish, or a combination thereof. The tag may comprise an internal component and/or an external component. RFID tags may come in any variety of shapes and sizes. The tag is preferably small and easily inserted into a fish. A small tag is one less than about 15 mm long and less than about 8 mm wide. The tag is preferably non-toxic to the fish and to any predator (including human) that might latter catch and eat the fish. More preferably the tag is a color that would enable a person Cleaning or eating the fish to find and remove the tag prior to eating it.

The tag may include any information acquisition or transmission technology. The tag may include a radio frequency identification (RFID), barcode, magnetic stripe, voice data entry and/or other automatic identification technologies.

RFID technology includes a sensing device (tag reader) which transmits a radio frequency signal to a specially designed RFID tag, which responds with another radio message. The RFID system may include the RFID active or RFID passive transponders (the tag) and the RFID reader. The RFID reader emits a frequency magnetic (electromagnetic) field via its antenna. When a RFID transponder passes within range, it is excited, causing it to transmit its data back to the RFID reader. Transmission and reception may occur nearly simultaneously.

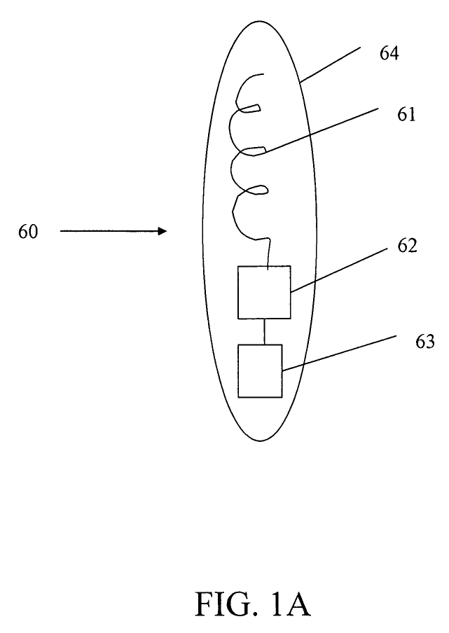

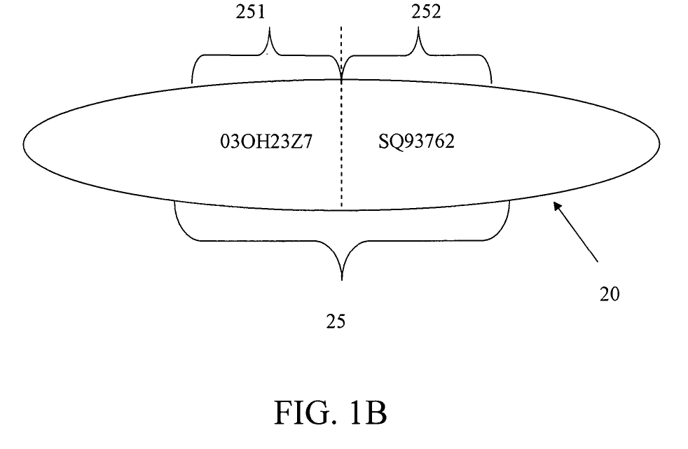

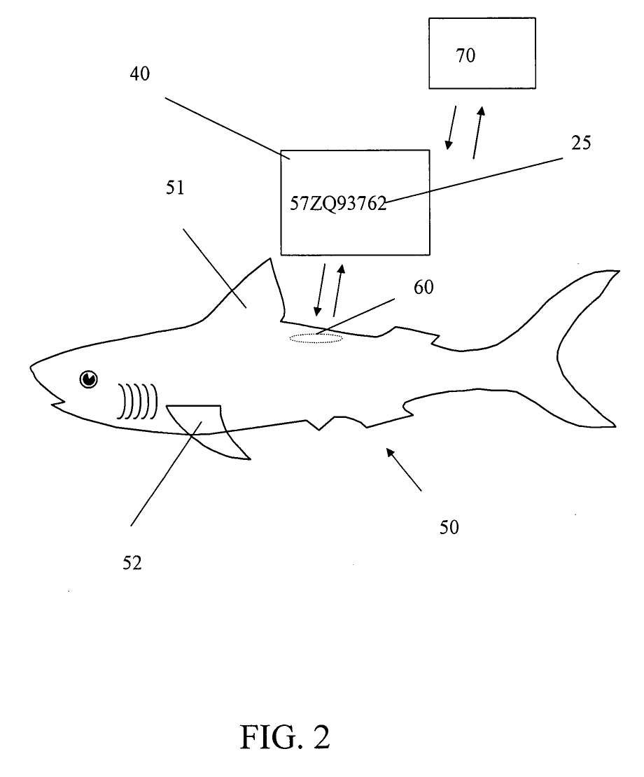

As shown in FIG. 1 the RFID tag 60 may include an antenna 61, a transceiver 62, and a transponder 63 electronically programmed with the identification sequence. The tag components, including the antenna 61, transceiver 62 and transponder 63 may be incased in a casing 64. The casing may be any material known in the art. The antenna 61 emits radio signals to activate the tag 60 and read and write data to it. The antenna 61 acts as a conduit between the tag 60 and a tag reader 40 shown in FIG. 2 (AKA a transceiver/decoder). The antenna 61 may be available in any variety of strengths, shapes and sizes. The tag 60 may be packaged with the tag reader 40 (FIG. 2).

RFID tags may be categorized as either active or passive. Active RFID tags are powered by an internal power source such as a battery. The active RFID tags are typically both readable and writable. They can have data read from them or written (added) to them. The data may be rewritten and/or modified. An active tag's memory size may vary between about 1 kilobyte and about 2 megabytes. An active RFID tag may be used to add information about the fishes capture to the RFID tag, in addition or instead of adding the information to the internet accessible data base. This encoded data added to the tag would then become part of the RFID tag data immediately available when the fish was caught again.

Passive RFID tags operate without a separate external power source and obtain operating power generated from the reader. Passive tags are consequently much lighter than active tags, less expensive, and may offer a very long (many years) operational lifetime. The trade off is that they have shorter read ranges than active tags and require a higher-powered reader. Read-only tags are typically passive and are programmed with a unique set of data (usually 32 to 128 bits) that cannot be modified. Read-only tags most often operate as an identification sequence into a database. Linear barcodes similarly reference a database containing modifiable information. The general uniqueness of each identification sequence correlated or linked to data in a database controlled by a company, individual, or service provider. Unknown individuals do not have access to update or change the data provided. The addition of a secure component to the identification sequence may be used to enable people unassociated with the initial fish tagging to act as a trusted party and add information into the database, if they obtain the complete identification sequence. The person is trusted to add data on that fish because that person has the entire identification code. While not everyone that catches a fish will enter data truthfully, in general the majority of the information obtained may be accurate and reliable.

RFID systems are also distinguished by their frequency ranges. Low-frequency systems operate between about 10 KHz and 750 KHz. Low-frequency systems typically have short reading ranges and lower system costs. High-frequency RFID systems typically operate above 500 MHz. High-frequency RFID systems offer read ranges greater than 50 feet and high reading speeds. High frequency RFID systems may be used however, the high-frequency RFID systems may be more expensive that lower frequency systems.

One advantage of the RFID systems is the noncontact, non-line-of-sight nature of the technology. The tag may be read through the body of the fish by a tag reader. In other embodiments, the tag may even be read through a portion of the water, reducing the need to remove the fish from the water. Depending upon the application, passive, low frequency tags may be preferred to avoid the targeting of previously caught fish by other anglers with high powered readers.

External Indication

FIG. 1B shows an external tag 20, that includes an identification sequence 25. The particular identification sequence 25 shown is 03OH23Z7SQ93762. The identification sequence 25 includes a public sequence 251 and an access sequence 252. The particular public sequence 251 shown in 03OH23Z7. The particular access sequence 252 shown in SQ93762.

An external tag, visible on the fish, may be used in the place of an internal tag, or in addition to an internal tag. The external tag may include the identification sequence. Alternatively, the external tag could be in addition to an internal tag with a readable identification sequence. An external indication of the tag may help avoid the inadvertent double tagging of a fish. It may also help avoid taking the time to scan a fish that is not already tagged. One disadvantage to an external tag is that it may snag on underwater obstacles and may be a source of irritation or infection to the fish. An external tag is also susceptible to falling off over time. Generally, an external tag may be most preferable for fish large in proportion to the tag, and/or fish that are frequently caught. Bar codes with an identification sequence with the public sequence and the access sequence may be used on an external tag.

Tag Reader

FIG. 2 shows a tag reader 40, reading a tag 60, in a fish 50. The fish has a dorsal fin 51 and a pectoral fin 52. The tag reader 40 emits radio waves which may travel and be easily detectable anywhere from about 2 centimeters (cm) to about 35 meters (m) or more, depending upon its power output and the radio frequency used. When an RFID tag 60 passes through an electromagnetic zone created by the radio waves, the tag 60 detects the tag reader's radio wave activation signal, activates using the energy from the radio wave and transmits the identification sequence 25. The tag reader 40 is designed to be used to obtain information such as the identification sequence 25 encoded into the tag 60. The data may be read, transmitted or otherwise entered into a computer 70 for processing. The transfer to a computer 70 may occur during the tag reading process, or afterwards and/or elsewhere. The data may be stored in the computer by any means known in the art. The tag reader 40 may be configured as either a handheld or a fixed-mount device. The tag reader 40 may be powered by any means known in the art including solar and/or battery power. The tag reader my include a computer and/or other data recording componenet.

Preferably, the tag is read by a tag reader that will detect and/or record the tag identification sequence. More preferably, the tag reader will also record other data to minimize the amount of data reentry required. For example the tag reader (sensor) could be programmed and/or combined with other devices to provide the fish weight, length, time of day caught, Global Positioning System (GPS) location, water temperature and the like. This data could them be downloaded for inclusion into the data set and/or database being maintained. The information may alternatively be recorded by the angler by any means known in the art including pencil, pen, palm pilot, or computer, for later entry into the data set and/or database.

The tag reader may also be connectable to a GPS device, depth finder, temperature gauge, wind gauge, speed gauge, scale, or other fishing aid to allow downloading of the data to the reader, computer or other device for later entry into the database. Even more preferably, the tag reader could be used to store data about the fish, and this data could be entered electronically (digitally) downloaded into the Internet accessible database.

Information

The internal and/or external tag should include the identification sequence. The identification sequence may be established by any method and/or process known in the art. Suitable identification sequences may be alphabetical, numerical, alphanumerical, detectable signal(s) or other symbol combinations. Detectable signals may be converted to identification sequences such as alphanumerics for simplified data recording. The identification sequence can include any number of characters. The each identification sequence may preferably be random and unique. Alternatively, not every identification sequence may be unique. The particular fish information such as species and general location of the tagged fish may provide sufficient detail to allow some repetition of particular identification sequence. Other information in addition to the identification sequence may be obtainable from the external tag and/or the internal tag.

The identification sequence 25 (FIG. 1B) may be used as part of the data entered into a database. The identification sequence 25 may be in one or more parts. For example, a portion of the identification sequence 25 may be a public sequence 251 (FIG. 1B) for public identification and dissemination. The public sequence 251 would identify the fish, either by itself, or in combination with other information such as the fish species, or location.

A second part of the identification sequence may be an access sequence 252 as shown in FIG. 1B. The access sequence 252 is used to identify a person that either has been, or is, in possession of the fish tagged with the tag containing the complete identification sequence. The access sequence 252 is the portion of the identification sequence 25 required for an individual to add and/or update one or more pieces of information (data and/or dataset) on the fish in the database.

The access sequence 252 may be part of the data entered into the Internet database but it should not be readable or accessible by someone accessing the data on the Internet. The access sequence 252 is designed to ensure that people without the complete identification sequence 25 do not enter fraudulent data and/or fraudulent dataset into the database. Those that catch the tagged fish may obtain the access sequence and update and/or add data and/or a dataset to the database. The public sequence 251 may also be used to identify a specific fish, and/or correlate specific data to a specific fish. The public sequence 251 would preferably allow read only access on the Internet. Preferably, each fish would have a unique access sequence and/or public sequence.

In one example, a fish may be tagged with the identification sequence 45OH30004Q-9001. The data recorded in the database included:

Identification sequence: 45OH30004Q-*********(* indicates the access sequence is not visible on the computer monitor or other display); Fish species: large mouth bass; Weight 3.2 kg, Length: 45 cm, Bait used: Rat-L-Trap® Bleeding Shiner; Date: Mar. 13, 2002; Time: 8 am; Location: Rocky Fork Lake, Ohio; GPS location: unknown; Water Temperature: 5 degrees C; Water depth: 5 meters; Anglers e-mail: 4jeffm**re@*n**net.com. Do you want to be contacted if this fish is caught again: yes; Other information: One of 8 fish caught and tagged that day. I caught this one along the rocks on the south shore east of South Beach. Fishing has been good for the last couple of weeks.

About two years later, the fish may be caught again. The angler or other user may record the identification sequence and details about the fish. Later the angler logs onto the Internet. In order to enter data on a fish previously caught a second angler or other user must enter the correct access sequence. The second angler entered the following data:

Identification sequence: 45OH30004Q-*********(-*********(* indicates the access sequence is not visible); Fish species: large mouth bass; Weight 5.2 kg, Length: 54 cm, Bait used: night crawler; Date: May 10, 2004; Time: 1 pm; Location: Rocky Fork Lake, Ohio; GPS location: unknown; Water Temperature: 15 degrees C; Water depth: 3 meters; Anglers e-mail: NFR. Do you want to be contacted if this fish is caught again: yes; Other information: He is still there. My eight-year-old daughter caught him while we were fishing for crappies. I would have kept it, it was the biggest fish she had ever caught, but when we scanned it and found the tag we decided that it wasn't really “just ours.” I sure hope the next guy lets it go so we can read about it. It was a great day. P.S. we did eat a few crappies

[symbol]

Since the first angler included his e-mail, the data was sent directly to the first angler about his fish. The second angler either did not want his e-mail address on the web, or planned to change it. An NFR, or similar entry may stand for “NOT FOR RELEASE.” In this instance, at least the public sequence of the identification sequence of the fish would be linked through the Internet to the second anglers e-mail. The second angler could automatically get e-mail from the web site, whenever the data on a fish he was interested in was updated. This type of arrangement may require a fee, or an agreement to also receive e-mail from businesses that sell fishing or other outdoor equipment. The angler or other user may have a different user enter and record the data and/or data set about their fish. Typically, the first angler or user and the second angler or user are different individuals without a prior relationship. It is of course possible for the same person to catch the fish a second time.

As shown above, it may be desirable for the identification sequence to have a portion that is not kept secret. This would simplify some aspects of data sorting, should the data be published in a written format such as a book or magazine. The access sequence may also facilitate links to specific e-mail addresses or web pages. For example, identification sequence 45OH30004Q-9001 may read 45OH30004Q-****** (* blank) on the Internet. Both the public sequence and the access sequence may be unique so that someone looking for a specific fish in the public database could search by the public sequence. The information is enterable into the Internet database by those possessing both the public sequence and the access sequence for a specific fish. The published part of the identification sequence could include data as well. The public sequence above could indicate that the tag was manufactured in 2004 and sold in Ohio. The data could also be recordable into a database and used on a computer without the Internet.

Upon entering the complete identification sequence an introductory page such as the following could be displayed on the Internet web page:

Congratulations, you caught one of 2,365 fish tagged to track the fish released in Rocky Fork Lake in 2008. Our goals include learning more about fish habits, their survival, and to encourage the practice of catch and release fishing by Ohio anglers. The state fisheries department and several local businesses contributed their time and money to this project. Please provide as much information about your fish and about yourself as possible so that we can continue to provide angling opportunities at Rocky Fork and around the state. Upon completion of this information, you may be e-mailed a coupon good for 10% off your next purchase from any of the business that contributed to this effort. It is our way of saying thank you, and encouraging everyone to participate in this program and practice catch and release fishing.

Over time, the information gathered may provide information on both fish and anglers. The information on fish may include growth rates, mortality, travel, density, and the like. Information on anglers may include their total fish catch, where they fish, who they are, what equipment they use, their conservation activities and the like.

Medication

There are few opportunities other than the capture of a fish to apply a health helpful substance directly to wild and semi-wild fish. The fish tagging process may provide an opportunity to apply a health helpful substance including medication, immunization, vitamins, and/ or combinations thereof to the fish. The tag may preferably include a substance helpful to the health of the fish such as at least one medication and/or vitamin. When the tag is inserted, the health helpful substance is also applied to the fish. Combining the helpful substance with the tag minimizes the number of fish body insertions required and the time required to tag and immunize the fish. The health helpful substance may be in the tag or coated about the tag. Alternatively, the tag may be contained in a liquid solution that includes the health helpful substance.

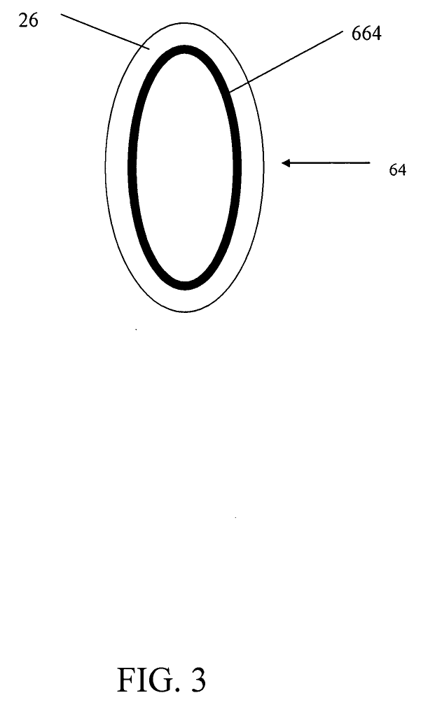

FIG. 3 shows the tag 64 with the casing 664 and a health helpful substance 26 applied to the casing 664. Preferably, the health helpful substance 26 may be absorbed through the tissue of the fish. The health helpful substance 26 may also help heal any wound in the fish resulting from the insertion of the tag. The health helpful substance 26 may include an antibiotic, vaccine, anti-fungal, anti-viral, anti-parasite, antibacterial, immunization, vitamin and combinations thereof. Examples of antibiotics include erythromycin, minocycline, tetracycline, penicillin, and sulfonamides. Sulfonamides include sodium sulfathiazole, sodium sulfamethazine, sodium sulfacetamide, and the like. MARACYN® and MARACYN II® by Mardel are examples of an antibiotics. Anti-fungal's include MAROXY® by Mardel, neutroflavine and the like. Anti-parasite substances include copper sulfate, trichlorfon, metronizadole, and the like. The tag may also include a sedative to relax the fish after capture. Any health helpful substance 26 may be customized for a specific fish species and/or size.

A surface treatment such as a cream or ointment may also be applied to the fish at the point of tag insertion to minimize harm to the fish. Two exemplary creams include DEBRIDE® and ORABASE®. Iodine, potassium permanganate, and/or hydrogen peroxide may also be used. Any combination of the above substances may be used, depending upon the particular desired application.

The Tagging Process

The tagging process is the process by which the tag is affixed to the fish. The tagging process may be by any means known in the art. The tagging process preferably causes minimal harm to the fish. The tagging process is designed to be done in a manner that minimizes any harm to the fish and allows the fish to be released with a reasonable expectation of survival. The fish should be handled as little as possible. Basic fish information may be gathered either before or after the fish is tagged. This information may include the fish length, weight, girth, and the like. The fish may be scanned to ensure a previous tag has not been attached internally or externally to the fish. The scanning may be visual, physical or by a tag reader. If no other tag is located, a tag may be attached to and/or inserted in the fish. Most fish are small enough that an internal tag may be inserted anywhere and it will likely be found easily by a scanner. For larger fish, a specific insertion point may be desirable and specified on the web page and/or pages associated with the tag. For example, fish over about 15 cm long may have the tag inserted near the port side of the dorsal fin.

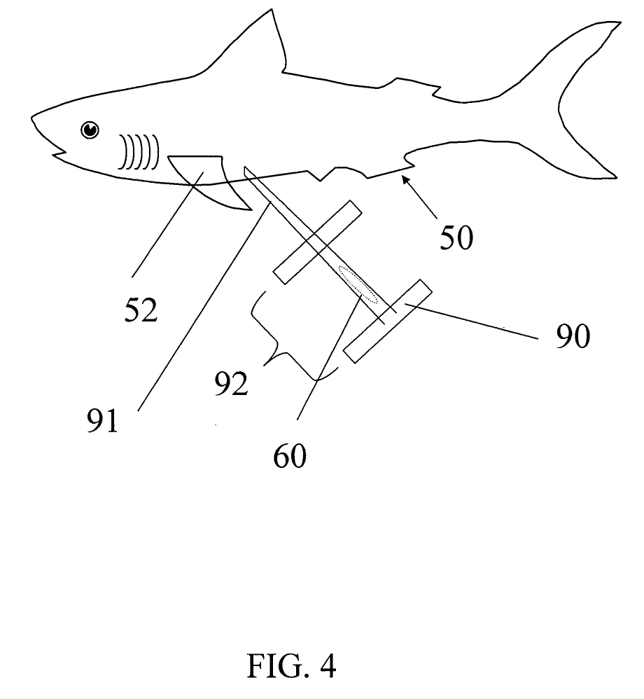

As shown in FIG. 2, the tag 60 may preferably be inserted into the fish 50 at the base of the dorsal fin 51. Alternatively, as shown in FIG. 4, the tag 60 may also be inserted into the fish 50 just behind the pectoral fin 52. An insertion tool 90 may be used to insert the tag 60. The insertion tool 90 may be any tool known in the art. The insertion tool may include a needle 91 and/or syringe 92. Similar devices such as pumps may also be used.

The Fish Source

The fish may be hatched in captivity or captured by any means known in the art including a fishing pole, net, electric shock etc. The fish is preferably wild but may be pen raised. The fish may live in a fresh or salt-water environment.

The Data Base

One component of the business method may be a widely accessible database of fish and other data. One method of providing wide access includes an Internet web access point “web page” that is searchable and updateable by the general public. This database may be directly updateable or verified and entered by a web manager. Preferably, the public sequence 251 and the access sequence 252 controls will be sufficient to limit inappropriate use and misinformation being submitted to the database.

The database is designed to include at least one data set on at least one fish. The database is designed to be sortable, searchable, expandable, and/or changeable. It is anticipated that as fish are tagged and released the database may eventually hold information on many thousands of fish.

The data in at least one data set for a specific fish may be added, changed or updated by individuals or organizations in possession of the complete identification sequence (access sequence 252). Preferably this data set input may be made by computer using the Internet. More preferable this data input may be made over the Internet. Preferably, the data set may be added to the database, but previous data and or data sets may not be deleted. Preferably, any one data set may not be updated more than once a day.

The database may function utilizing any communication protocol. The Defense Advance Research Projects Agency (DARPA) originally developed Transmission Control Protocol/Internet Protocol (TCP/IP) to interconnect various defense department computer networks. The Internet, an international Wide Area Network, uses TCP/IP to connect government and educational institutions across the world. TCP/IP is also in widespread use on commercial and private networks. Communication protocol may include Hypertext Transfer Protocol (HTTP), Secure Hypertext Transfer Protocol (HTTPS), File Transfer Protocol (FTP), Wide Area Information System (WAIS) (supports retrieval of documents from databases via full-text search) and/or any Uniform Resource Locator (URL) recognizable by a browser such as Netscape or Windows Explorer. The web site www.protocols.com lists dozens of available protocols for particular applications.

The database may be maintained by a commercial entity, not for profit entity, a wildlife service organization, a guide service, or an individual. Data entry preferably adds to the data from the previous fish tagging or release. This allows a history of the fish to be maintained as each new capture and release of the fish adds to the data already recorded. As a result, after multiple captures multiple fish weights and other information may be contained in the database.

The data gathered by this approach may also provide useful information on both the fish and the anglers that catch them. The data may encourage the release of fish through participation, peer pressure, and an increased desire to help the fish population. Released fish provide future opportunities for that fish to be caught again. It also provides that fish an opportunity to breed and produce offspring which may also be caught another day, or feed other animals in the ecosystem.

Information and statistical data obtained from the database may help assess fish populations, locations, mortality, habits, growth rates and the like. The fish information may be particularly helpful on lakes where slot fishing is practiced. Slot fishing occurs when fish of a particular species and size are required by the fishing regulations to be released. Sometimes this is a minimum size but it may also be a mid-range size between a specified weight and/or length limit, thus the term “slot fish.” The slot fish that must be returned may represent the preferred fish breeding age. The tagging of slot fish may help encourage compliance with the release requirements. It can be frustrating to fish all day and catch numerous fish in the slot that cannot be kept for a fish dinner. Catching tagged fish let's the angler know that he was not the first one to catch the fish in the slot and that without the release requirement he would be catching fewer fish overall. This feedback may help build “buy-in” or acceptance by the anglers that the slot requirements are necessary for a healthy fish population. This buy-in may also make it easier to expand slot-fishing regulations to other waters that could benefit from such a restriction. Gaining the buy-in of recreational anglers for new fishing restrictions in the interest of long-term resource management is an ongoing challenge for Federal, State and local fish management organizations. Tagging may also help encourage the release of otherwise legal fish which were previously tagged. Information on the survivability of caught slot fish and/or their growth rate may be helpful in determining the optimum size for the slot-fish. The information may also help identify other conservation efforts that may be helpful to the fish population.

Information on the anglers may include where they are from, their success, travels, spending, equipment, conservation activities, identity, and the like. This information may help State and/or Federal agencies monitor the fishing success of a particular lake or region and implement policies that help both anglers and the fish population. Companies may use the information to better target their marketing efforts. Anglers may also use the information to discuss fishing, find new fishing partners and/or learn more about the fishing in their area.

The Internet structure of the present invention may preferably include at least one web site entry point to access the information on the database. The web site may be used for recording at least a portion of the tag identification sequence and at least a portion of the fish information. The web page could be located at for example, www.fishid***.net. Alternatively, the entry point may be the web page of a prominent fish research center, a web page dedicated to a particular species of fish, or angler supply store. Once on the web page, the web page may include a search routine, banners, sponsors and discussion groups. A web visitor may be able to search by state, fish type, lake, specific angler or any other data group within the database. In one embodiment, the data may be sortable or cataloged by a particular resort, agency, or guide service. The data could also be maintained by a guide service on a personal computer for their own use, although this type of database would lack many of the advantages of the Internet accessible database disclosed herein.

Preferably, at least a portion of the identification sequence (ID, fish ID, or code) is not viewable or discernable by a user on the Internet. In order to update or add data (or a data set) on a particular fish, the access sequence (complete identification sequence) must be known. This limits the number of people capable of falsely inputting data about a particular fish. A person could search a particular lake in a particular state for all the recorded large mouth bass. However, that individual could not update that bass's information without the access sequence component of the complete identification sequence for a given fish. When entering information or a data set on a particular fish, the access sequence would have to be provided. After a certain number of failures, the system may be designed to lock out the person and/or computer entering invalid identification sequences. Preferably the public sequence would be used to match the access code with the specific data or data set in the database. A particular access sequence would only allow access when entered in conjunction with the correct public sequence. Many different public sequences and access sequence combination may be used to ensure the capture of one fish would not allow the changing of multiple fish data sets.

One advantage to having a public sequence and an access sequence is the ability to share consistently and easily identified data while maintaining confidence in any new data and/or data sets added to the database. Tagging data can be shared with other interested organizations without the access sequence. Any conclusions made based upon the data can be correlated with the public sequence and independently verified.

For example, one identification sequence may be AX345G9-I9476 attached to a bass first caught in Bass Lake, Minn. In order to update a data set of information about this fish, the fish species and identification sequence be provided by a subsequent angler. A help-line could be available to override this limitation. For example, if the fish was a largemouth base and the original data entry identified it as a small mouth bass in error. Preferably, when an identification sequence and other data match is provided, the data entry fields are presented with the prior fish information already complete. Only that information that needs changing would have to be entered by the person entering the data. For example if the fish weight was 8 kg and is now 10 kg, that data may be up-dated and/or added with another data set of information about the fish and other desired data. The original data set on the fish may remain unchangeable, or be unchangeable after a certain number of days subsequent to it's entry into the database. Thus when a fish is caught, new information may be added, but old information cannot be deleted or altered.

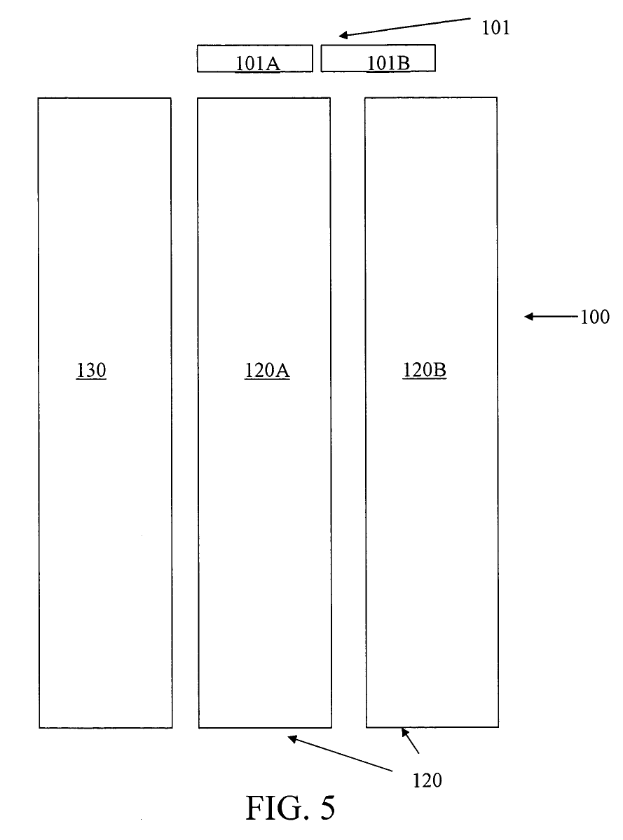

By way of a non-limiting example, FIG. 5 shows information on a fish with a possible data entry screen 100 containing non-limiting date fields 120. As shown in FIG. 5, this information may include a portion of the identification sequence 101. The identification sequence 101 shows the public sequence 101A data entry field. For a new data set entry, the public access field may be blank or pre-filled if the fish had been previously captured. There may be a blank data entry field for the access sequence 101B. When the correct access sequence is entered, an additional or new data and/or data set 120B on that fish may be added to the database and/or made available for data entry. The data sets 120A and/or 120B may also be changed. Alternatively, this data in data set 120A could become unchangeable by an Internet user after a certain period of time. For example, data entered would not be changeable by a user 2 days after entry into the database. This may help limit the ability of one user changing the data entered on a fish by a previous user. Data set 120A may be data for the fish when it was first caught (e.g.on about 3/13/×2). Data set 120B is for the fish when it was later caught (e.g. on about 7/4/×4). Each data set may have multiple data fields for specific information. FIG. 5 shows several possible data fields 120 for subsequent data sets 120A, 120B, and others as needed (not shown). Data set categories 130 identify the appropriate data type the be entered in each data field of the data sets 120A, 120B. Data set categories 130 may identify data fields 120 for data such as the Identification Sequence, date caught, time caught, weather details, water temperature, fishing depth, fish species, year caught, date caught, time caught, fish weight when caught, fish length, location caught, angler information, water details, lure used, other data, a picture and the like. Further location information may include the approximate Global Positioning System (GPS) location, State caught or body of water. Angler data includes such items as their name, e-mail address and whether they would like to be automatically notified by e-mail when the fish is caught again. Other data fields of information may be provided as requested by anglers and desired by anglers and those using the database for research purposes. Other information could include the number of fish caught that day, the total number of fish released by that angle for the year, and the like. A general field for other information may also be provided. A link to other web sites and/or a picture may also be provided.

Preferably, the person entering the data may indicate whether they have a desire to receive e-mail whenever this fish is caught again. This enables an angler to receive feedback on the survival and growth of “their” fish. These reports back to the anglers that previously caught and released the fish may be preformed by the angler, the Internet site manager, automatically through software coding, or by the resort and/or guide service used by the angler. The resort of guide service may provide information on the fish and use the opportunity to suggest to the angler that they return for another fishing trip in the future. The database on the Internet is preferably sortable by at least one data field 120. For example, a person could list all the large mouth bass tagged, or more specifically all the large mouth bass from Rocky Fork Lake tagged in the year 20xx. The sort may narrow the search with as few or as many fields desired.

In addition to a widely accessible internet database, the database may be maintained locally on a personal computer by resorts, guide services, and the like. For example, resorts and guide services could maintain their own database. When the fish is recaptured, it may provide an opportunity and/or reason for the resort and/or guide, service to notify the previous anglers that caught that fish. The resort and/or guide service may suggest that the angler return for another visit to fish. It would also help the resort or guide service protect the resources they market. The advantages of promoting catch and release may be significant enough to a resort that they would do the record keeping, data entry and fish tagging. The resort and/or guide service may even pay for the tagging and release of any fish over a certain weight. Resorts and/or guide services that depend upon the fish resources of a particular waterway are highly motivated to protect the fishing resource and may be willing to use and support this method as one tool for encouraging the release of fish, especially trophy fish.

The database may include marketing information as a component of the data maintained for public access. For example, a web site, an identification sequence and/or group of identification sequences may be associated with a sponsor or other entity that is supporting the tagging, and release of fish. The sponsor may be a business, a federal organization, state organization, non-profit organization, conservation organization, resorts, guide services and the like.

For example, in order to increase the fishing success of a particular lake, federal agencies, state agencies, local agencies and/or businesses may donate funds for tagging 2,000 fish. At least a portion of the identification sequences in the tagged fish may correspond to data sets in the database. Included in the data set associated with the public sequence and/or access sequence of the identification sequence may be marketing information from the various tagging sponsors. For example, when an angler enters the access sequence on a fish, a marketing screen may appear (pop-up) on the web site. The screen may be similar to the typical pop-up screen used on the Internet today. The marketing screen may include information such as:

Congratulations, you have caught one of 2,360 fish tagged to encourage the catch and release of fish in our area. Yours was the 236^{th} fish tagged. The State Fisheries Department and six local businesses contributed their time and money to encourage the conservation of this natural resource. Please update as much information on your fish as possible on this web site. The data being gathered will help encourage the release of fish and provide information to our State Fisheries Department that may help further improve the fishing in our area. Thank you for your participation in this fish management effort. If you print this screen and bring the printout with you to Bills Bait and Tackle. you can receive a free fishing lure valued up to $5.00.

The data gathered and recorded in the database may be categorized, sorted, analyzed, cross-referenced and/or manipulated to provide statistical information on fish and/or the anglers that catch them. This analysis may be maintained on the Internet, typically accessible from at least one web site. The data and information may alternatively be periodically published as a document in a book or magazine form. Alternatively, the database may also be downloadable from the Internet into a formatted document. Preferably, the user may specify the data set organization and structure. The published document could be sold and/or given away for use as a marketing tool. The marketing users may include resorts, guide services, and other organizations that want to inform previous and/or potential customers about their fishing success, and their efforts to protect the fishing resource.

RFID tags, particularly high frequency RFID tags may have particular utility with larger fish. For example, some sailfish are tagged and released. In order to subsequently remove and/or read the tag, the fish must be brought close to the boat, typically within a meter or two of the boat. Getting the sailfish this close to the boat may require exhausting the fish. The last few meters to the boat may be particularly exhausting and/or traumatic for the fish, possibly causing it to expend energy reserves it may need for long term survival. A high frequency RFID, and/or a high powered tag reader may be used to read the tag further from the boat, thus causing less stress on the fish. The tag could be read and the fish released without forcing the fish in close proximity of the boat. A database of tagged fish data could be used to extrapolate the present size of the fish from the original tagged fish weight, tagging date and/or other parameters. Tournament and guide services could release the fish early if the extrapolated data indicated the fish was not likely to place in the tournament or meet the clients trophy goals. This may save the fishermen time better spent on bigger fish and save the fish valuable energy it may need for it's continued survival.

The present invention has been shown and described herein in what is considered a practical and exemplary embodiment. It is recognized, however, that departures may be made there from within the scope of the invention and that obvious modifications will occur to a person skilled in the art.

We claim:

1. A method of doing business on an Internet for at least a first user and a second user to identify and update information on at least one fish wherein:
an article with an identification sequence is applied by the first user to the fish prior to the fish being release;
at least a portion of the identification sequence is used to identify the fish,
at least a portion of the identification sequence is used to identify the second user as a subsequent possessor of the fish;
a database accessible from the Internet includes a first data set containing information on the fish and at least a portion of the identification sequence; and
the second user uses at least a portion of the identification sequence to obtain access to change at least a portion of the database.

2. The method of claim 1 wherein at least a portion of the identification sequence is not ascertainable on the Internet.

3. The method of claim 1 wherein the second user obtains the identification sequence and uses at least a portion of the identification sequence and the Internet to add data to the database.

4. The method of claim 1 wherein the second user uses the identification sequence to add a second data set on the fish to the database.

5. The method of claim 4 wherein at least the second user receives a reward for adding the second data set on the fish to the database.

6. The method of claim 5 wherein the reward is a coupon, gift certificate, or product.

7. The method of claim 1 wherein the database includes data on more than one fish.

8. The method of claim 7 wherein at least a portion of the database may be searched and sorted.

9. The method of claim 8 wherein the database may be searched and sorted by a third user on the Internet.

10. The method of claim 1 wherein the database is downloadable from the Internet to a local storage medium.

11. The method of claim 1 wherein the first user and the second user are different and unknown to one another.

12. A method for tracking a fish wherein:
an identification article with an identification sequence is applied to the fish;
at least a portion of the identification sequence is used to identify the fish;
at least a portion of the identification sequence is used to gain access through the Internet to at least a a portion of a database; and wherein
the access includes the ability to edit the database.

13. The method of claim 12 wherein the accessible portion of the database is limited to adding a dataset.

14. A method for encouraging the release of a fish caught by a fisherman including:
Attaching to the fish an article with an identification sequence;
recording at least the identification sequence and at least one characteristic of the fish;
releasing the fish;
accessing at least a portion of a database;
storing at least a portion of the identification sequence and the at least one characteristic of the fish as a data set in the database;
recapturing the fish;
accessing at least a portion of the database; and
adding at least one updated characteristic of the fish to the data set in the database.

15. The Method of claim 14 wherein the database is accessed through the Internet.

16. The method of claim 15 wherein the Internet includes at least one website with at least one web page; whereby the web page includes banners, links to other websites, or advertisements.

17. The Method of claim 14 wherein releasing includes returning the fish to a body of water such that the fish has as much freedom of travel as it had before being caught.

18. The Method of claim 14 wherein the method includes:
recapturing the fish by a second fisherman;
scanning the fish with a reader to determine the identification sequence;
acquiring at least one second characteristic of the fish;
releasing the fish a second time;
accessing at least a portion of a database using the identification sequence;
adding the at least one second characteristic of the fish as additional data to the database.

19. A method for tracking at least one fish comprising the steps of:
tagging the fish with an identification sequence comprising a public sequence and an access sequence;
recording at least one piece of data about the fish in a data set,
placing the data set in an internet accessible and searchable database such that the dataset is relatable to the public sequence;
allowing a subsequent possessor of the fish to obtain the identification sequence and use the access sequence to add at least one second data set to the database, the second data set is also relatable to the public sequence.

20. The method of claim 19, wherein the fish is tagged with an external indicator.

21. The method of claim 19, wherein the database uses a software based communication protocol.

22. A fish identification tag including:
an identification sequence having;
a first indicia for public access and dissemination to facilitate identification of the fish and differentiating the fish from other fish;
a second indicia with limited access to identify individuals in possession of the tag, the second indicia identifying the individual as a possessor of the tag and a legitimate source of at least one piece of data related to the fish; and
the second indicia providing limited access to edit an internet accessible searchable database.

23. The tag of claim 22, wherein the tag includes an antibiotic, anti-fungal, antiviral, or vaccine.

24. The tag of claim 22, wherein a reader is used to read the identification sequence.

25. The tag of claim 22, wherein the tag is disposed internal to the fish.

26. A fish tagging kit including:
at least one tag;
a tag attachment device;
instructions on how to tag the fish;
instructions for recording data; and
and a web address of an Internet accessible web site.

27. A method of creating an accessible and searchable database of information on fish, the method including:
at least one first user tagging at least one fish with a sequence identifier having a public sequence and an access sequence, the first user entering a data set about the fish into a searchable database;
at least one second user recapturing at least one fish and using the access sequence to enter at least a second data set of additional information about the fish into the searchable database;
the public sequence being used to link both data sets to the fish; and wherein
the database is obtainable for review by a third user.

28. An identification tag including an identification sequence,
the identification sequence having
at least one public sequence, and
at least one access sequence.

29. The tag in claim 28 wherein the identification sequence is generated by a radio frequency.
